(19) United States
(12) Patent Application Publication
(10) Pub. No.: US 2011/0125004 A1
(43) Pub. Date: May 26, 2011

(54) Analysis by photo acoustic displacement and interferometryl

(75) Inventors: Kiran Kumar Thumma, Eindhoven (NL); Bastiaan Wilhelmus Maria Moeskops, Eindhoven (NL); Golo Von Basum, Eindhoven (NL); Yan Liu, Eindhoven (NL)

(73) Assignee: KONINKLIKJKE PHILIPS ELECTRONICS N.V., EINDHOVEN (NL)

(21) Appl. No.: 13/055,531

(22) PCT Filed: Jul. 24, 2009
(86) PCT No.: PCT/IB2009/053219

§ 371 Date: Jan. 24, 2011

(30) Foreign Application Priority Data
Jul. 31, 2008 (EP) 08161518.9

(51) Int. Cl. A61B 5/05

(52) U.S. Cl. 600/407

(57) ABSTRACT

A device for analyzing a material, the device having a photo acoustic generator (40) for generating pressure waves in the material by pulsed illumination by a light source, and a sensor (20, 30, 50) for producing a signal using self mixing interferometry, the signal representing displacements of the material due to the pressure waves, and a signal processor (60) for processing the signal to analyze the material. This combination of photo acoustic stimulation and self mixing interferometry sensing helps reduce or avoid errors or distortions caused by sensor contact on the material, and can provide a better ratio of sensitivity for a given degree of compactness of the device.

### FIELD OF THE INVENTION

This invention relates to devices for analyzing a material using photo acoustic stimulation, and to corresponding systems and methods of manufacture or use.

### BACKGROUND OF THE INVENTION

Many efforts have been put in developing instruments for non-invasive measurement of physiological parameters in a human or animal body, such as for example glucose measurements, based on optical methods. Although these methods have proven to have sufficient sensitivity for in-vitro and/or ex-vivo glucose quantification, devices based on such currently existing techniques have not been successfully brought to the market. In the home environment, self monitoring of blood glucose is a vital element in diabetes management. Current monitoring techniques use the inconvenient and painful method of drawing blood through the skin prior to analysis. Therefore, new methods for self monitoring of blood glucose levels are required to enable more rigorous control of blood glucose in diabetic patients. Numerous attempts at non-invasive monitoring have been undertaken by various companies, mostly using spectroscopy. None of the companies have shown reliable glucose measurements due to various constraints. To date, no non-invasive technology for self monitoring of blood glucose has been given an FDA approval.

There are various techniques, such as near infrared spectroscopy, and photo acoustic spectroscopy, which are used for determining the glucose concentration non-invasively. For most of the non-invasive measurement devices, suitable measurement sites on the skin are necessary for reproducible glucose measurements. This mainly involves imaging the skin to find out the suitable position, for e.g. on the capillary, prior to the measurement.

Photo acoustics is a well established field and relates to the generation of acoustic waves by the pulsed radiation. In biomedical optics, a pulsed laser radiation is applied on to the sample, skin in this case, and the radiation penetrates deep into the tissue. The radiation is absorbed by the blood present in the capillaries and the blood vessels. Due to this absorption there is a pressure build-up in the vessels which results in a pressure wave. This pressure wave travels onto the surface of the skin resulting in a displacement which is proportional to the absorption coefficient of the absorber. These photo acoustic signals can be detected by piezo electric detectors or in a non contact way by interferometric detection. This can be used for imaging to a depth of few millimeters inside the skin. Such photo acoustic spectroscopy techniques can also be used to detect the glucose concentration.

The surface displacements can be measured using for example Doppler effects, or optical interferometer. From a practical point of view this configuration is large in size and expensive, because a large number of different optical components are used. Because of this, these devices can only be used in laboratory conditions.

Surface displacements of a sample arising due to displacements triggered by photo acoustics have been detected using interferometers. Payne et al (J. Biomedical Optics, 8, pg 273, 2003) have demonstrated the photo acoustic imaging using the interferometer.

Laufer et al, (Physics in Medicine and Biology 50, p. 4409, 2005) and EP1711101 shows the use of photo acoustic spectroscopy for the determination of analyte concentration. The photo acoustic effect causes the displacement of the skin and this can be detected using non contact interferometer as mentioned in the literature. EP1711101 shows controlling a light source to illuminate tissue below the skin with light that stimulates photo acoustic waves in the tissue. The light can have at least one wavelength that is strongly absorbed by blood. Signals generated by a transducer array responsive to the photo acoustic waves are used to provide a “photo acoustic” image of features below skin, and in particular, blood vessels.

### SUMMARY OF THE INVENTION

An object of the present invention is to provide devices for analyzing a material using photo acoustic stimulation, or corresponding systems or methods of manufacture or use.

A first aspect of the invention provides:

A device for analyzing a material, the device having a photo acoustic generator for generating pressure waves in the material by pulsed illumination by a light source, and a sensor for producing a signal using self mixing interferometry, the signal representing displacements of the material due to the pressure waves, and a signal processor for processing the signal to analyze the material.

This combination of photo acoustic stimulation and self mixing interferometry sensing helps reduce or avoid errors or distortions caused by sensor contact on the material, and can provide a better ratio of sensitivity for a given degree of compactness of the device.

Another aspect of the invention provides a method of analyzing a material, having the steps of photo acoustically generating pressure waves in the material by pulsed illumination by a light source, producing a signal using self mixing interferometry, of displacements of the material due to the pressure waves, and processing the signal to analyze the material.

Embodiments of the invention can have any other features added; some such additional features are set out in dependent claims and described in more detail below.

Any of the additional features can be combined together and combined with any of the aspects. Other advantages will be apparent to those skilled in the art, especially over other prior art. Numerous variations and modifications can be made without departing from the claims of the present invention. Therefore, it should be clearly understood that the form of the present invention is illustrative only and is not intended to limit the scope of the present invention.

### BRIEF DESCRIPTION OF THE DRAWINGS

FIG. 1 shows the interference signal in a self mixing laser diode, the upper trace is the movement of the mirror and the lower trace is the interference signal from the laser diode due to self mixing effect,

FIG. 2 shows a device using the laser diode for detecting the pressure waves generated by the sample,

FIG. 3 shows the laser diodes arranged in an array to detect the surface displacements of the skin, and

FIG. 4 shows a view of a method according to an embodiment.

FIG. 5 shows a flow chart of an algorithm for use in signal processing according to an embodiment of the present invention.

### DETAILED DESCRIPTION OF EMBODIMENTS

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention. References to a signal can encompass any kind of signal in any medium, and so can encompass an electrical or optical or wireless signal or other signal for example. References to a signal processor are intended to encompass any means for processing the signal, and can encompass a personal computer, a microprocessor, analog circuitry, image processing software and so on.

The present invention also includes a computer program product which provides the functionality of any of the methods according to the present invention when executed on a computing device. Such computer program product can be tangibly embodied in a carrier medium carrying machine-readable code for execution by a programmable processor. The present invention thus relates to a carrier medium carrying a computer program product that, when executed on computing means, provides instructions for executing any of the methods as described above. The term “carrier medium” refers to any medium that participates in providing instructions to a processor for execution. Such a medium may take many forms, including but not limited to, non-volatile media, and transmission media. Non-volatile media includes, for example, optical or magnetic disks, such as a storage device which is part of mass storage. Common forms of computer readable media include, a CD-ROM, a DVD, a flexible disk or floppy disk, a tape, a memory chip or cartridge or any other medium from which a computer can read. Various forms of computer readable media may be involved in carrying one or more sequences of one or more instructions to a processor for execution. The computer program product can also be transmitted via a carrier wave in a network, such as a LAN, a WAN or the Internet. Transmission media can take the form of acoustic or light waves, such as those generated during radio wave and infrared data communications. Transmission media include coaxial cables, copper wire and fiber optics, including the wires that comprise a bus within a computer.

In the description provided herein, numerous specific details are set forth. However, it is understood that embodiments of the invention may be practiced without these specific details. In other instances, well-known methods, structures and techniques have not been shown in detail in order not to obscure an understanding of this description.

### Additional Features:

Embodiments described show examples of devices having combinations of photo acoustics apparatus and detectors based on laser diode self mixing (LDSM). Embodiments can have any additional features as well as those features set out in the independent claims. Some additional features are as follows:

The sensor can comprise a laser diode for outputting light which is directed towards the material so that a proportion is reflected so as to mix in a cavity of the laser diode to form a frequency and/or intensity modulation, the sensor also having an analyzer for generating a signal representing the displacements by analyzing the frequency and/or intensity modulation.

It can have an optomechanical arrangement for scanning the sensor over an area of the material. It can have an array of sensors for simultaneously generating signals for an area of the material.

Another such additional feature is a near infra red spectroscope sensor for generating a further measurement of the material.

The sensor can be arranged to sense parts of the material below a surface of the material, or to sense parts of the material on a surface of the material.

The device can be arranged as a non invasive monitor for monitoring concentration of constituents of blood in a human or animal body.

The method can have the step of generating an image of the material. It can be used for non invasive monitoring of blood in a human or animal body, involving the step of processing the signal to determine the concentration of given constituents of the blood, such as constituents which can indicate glucose levels or other indications.

Some embodiments involve devices having a laser diode in a self-mixing mode for detecting photo acoustic signals generated due to absorption of laser radiation in tissue. Some advantages of such a device are its miniature size and contactless operation. It requires no contact with the skin and therefore eliminates all the complexities and interfering effects associated with a contact measurement. Some applications of such devices are as follows. The device can be embedded into an optomechanical skin interface for photo acoustic skin imaging. In addition to skin imaging, this device can also be used to non-invasively determine the concentration of analytes (e.g. glucose).

For the non invasive monitoring of glucose accurately, an opto-mechanical probe skin interface, as proposed here, is needed by most techniques. The devices shown can provide a solution where the problems related to the force can be reduced or removed thereby increasing the reproducibility and sensitivity of the measurements. Another area of application is in determining other skin properties (e.g. for use as a tool in monitoring effects of skin cancer, skin aging, etc.) by means of light.

Such devices can remove the negative interference effects associated with skin contact such as force variation, temperature changes, variation in skin mechanics etc, compared to conventional techniques such as near infrared spectroscopy. In such techniques the skin light is delivered via fibers to the skin surface. These fibers are typically in contact with the skin to get maximum coupling efficiency.

A further distinction is that the device can be made more compact than conventional devices. It can use multiple laser diodes in a matrix for imaging applications. This is not feasible with a conventional interferometer. The laser diode can be used as an imaging modality, for e.g., obtaining a suitable measurement position. For imaging, an array of laser diodes can be used. The image can help identify the area of interest, such as a blood vessel, capillary etc. An external energy source for e.g., a tuneable laser source, is used at multiple wavelengths on the area of interest and subsequently induced displacements are detected by LDSM interferometry for tissue analysis using for e.g., spectroscopic techniques. The device is extremely compact as it uses self-mixing effect that occurs in the laser cavity thereby reducing the complexities of a conventional interferometer. As the interferometer can be miniaturized in the form of laser diode, it is easy to arrange multiple laser diodes in a compact matrix form for imaging applications.

As an added benefit, other sensing modalities can be added to the device for e.g., NIR diffuse reflectance spectroscopy, OCT etc. These techniques can be used along with the LDSM photo acoustic spectroscopy, to increase the accuracy of glucose concentration measurements.

In using a laser diode in a self-mixing mode to detect photo acoustic spectroscopic signals to determine analyte concentration, a number of features can be provided:
1. Generation of pressure waves by pulsed laser radiation.
2. The pressure waves results in surface displacement. LDSM is used to detect the displacements.
3. The light from the laser diode acts like a probe beam on the skin surface. The displacement of the skin due to the pressure wave changes the intensity pattern inside the laser cavity. LDSM is used with modulation techniques to detect the surface displacements.

### FIG. 2, Example Embodiment

A simple way to construct an interferometer is to use the self-mixing effect that occurs in the laser cavity, when external light is coupled back to the laser cavity as shown in FIG. 2. The main components are the laser diode 30, pulsed laser 40, spectrum analyzer 50 and a computer 60 for analysis.

The pulsed laser source is aimed onto the material sample 70, e.g. in the form of skin, via a fiber 80, for generation of pressure waves. The excitation system for generating photo acoustic signals consists of a pulsed laser light source with a wavelength range between 680 nm-1064 nm.

The excitation system can consist of multiple fibers, which are interspersed among the Laser diode arrays which are used for detection. Laser diode self-mixing is the coupling of emitted laser light back into the laser cavity 20 after reflection from an external surface. This is detected using a photo detector 10. By analyzing the intensity or electric modulation induced by the optical feed back in the laser, displacements, e.g. vibrations of the surface or the velocity of the surface or the acceleration of the surface can be quantified. This same principle can be applied to detect the surface displacements (or velocities or accelerations) caused due to the photo acoustic waves generated by illuminating the skin with pulsed electromagnetic radiation.

The problem of needing laboratory size apparatus for non invasive glucose measurements can be addressed since LDSM can be implemented with much more miniaturization. The LDSM sensor has a laser diode and operates as an interferometer. The light reflected from many objects such as the skin is diffusive due to a rough surface. Therefore, only a fraction of light is available for the interference with the reference beam which is the laser diode cavity. Hence the intensity variation is hard to measure due to displacements which are of the order of half the wavelength of the probe beam. Small displacements can be measured by looking at the phase changes which are proportional to the displacement. These small displacements can be determined by modulating the frequency and/or intensity of the laser diode. The light from the skin surface mixes with the frequency and/or intensity modulated beam of the laser diode. Due to the displacement of the skin, the phase of the mixed beam changes. The difference of the phase of the mixed beam and the phase of the laser diode modulation gives the displacement of the skin surface.

### Laser Diode Self Mixing:

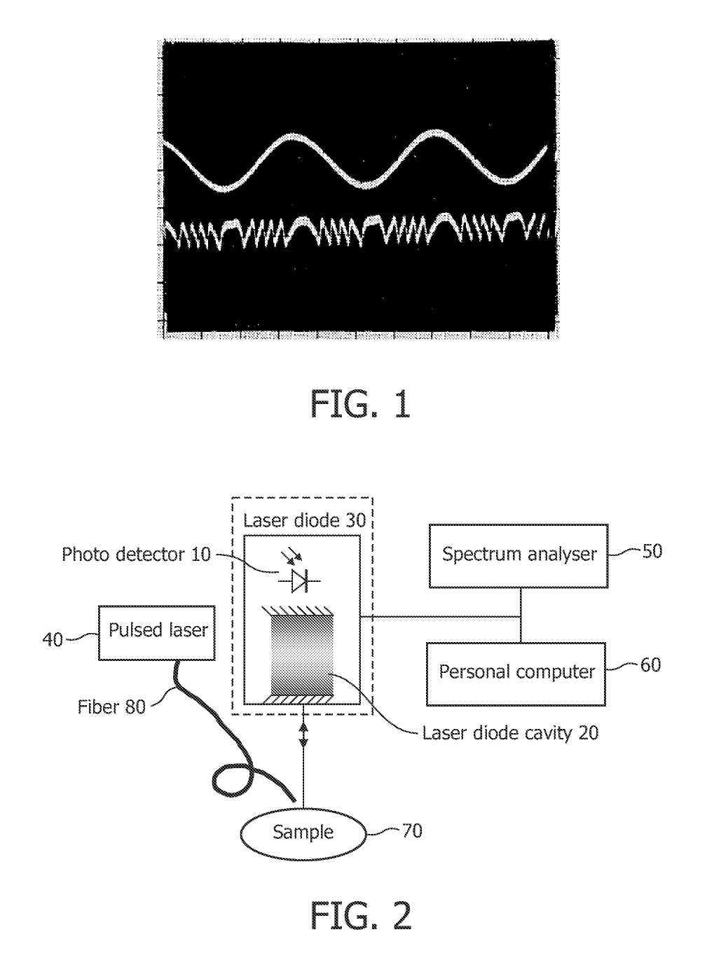

Since the approach of self-mixing was first reported by Rudd (J. Phys. E 1, 723-726 (1968)) by using the He—Ne laser to measure the Doppler velocity of scattering particles, the phenomenon of self-mixing or backscatter modulation has been investigated by several researchers. The FIG. 1 shows a typical interference signal obtained by the self mixing method. The upper trace is the movement of the mirror and the lower trace is the interference signal from the laser diode due to self mixing effect,

It is known that laser diode self mixing (LDSM) is used for remotely measuring speed, vibrations, range and length as described in U.S. Pat. Nos. 4,168,906, 6,233,045 and using an array of laser diodes in U.S. Pat. No. 4,927,263.

The above mentioned patents give the details of how the LDSM can be used to detect the vibrations or velocity of a surface. The device includes a laser diode that provides source light which is directed towards a surface whose surface vibration or velocity to be determined. The light from the surface mixes with the laser diode cavity and forms a frequency and/or intensity modulation. The velocity of the surface can be determined by analyzing the modulation. Takaaki Shibata et, al., (IEEE Tra. on Inst. and Meas, 48, 1999) measure both a velocity and a length of a moving plate, employing LDSM.

The implementation of the LDSM in the low feedback regime is also possible. The light reflection from rough surfaces forms a speckle pattern and a fraction of the incident light is available for the interference. The LDSM is also applied in the displacement of the biological samples. Jukka Hast in his thesis (OULU, 2003) describes the principle of LDSM towards the pulse detection.

These pressure waves cause a surface displacement on the skin. The laser diode acts as a probe beam on the skin. Due to the surface displacement, there is a change in the intensity modulation.

This modulation is detected by the photo detector, e.g. a photodiode present inside the laser diode. An algorithm is used to translate this intensity variation into the displacement and also to obtain an image from the displacement. The algorithm searches for the phase changes due to the surface displacements and analyses the intensity of the pressure waves.

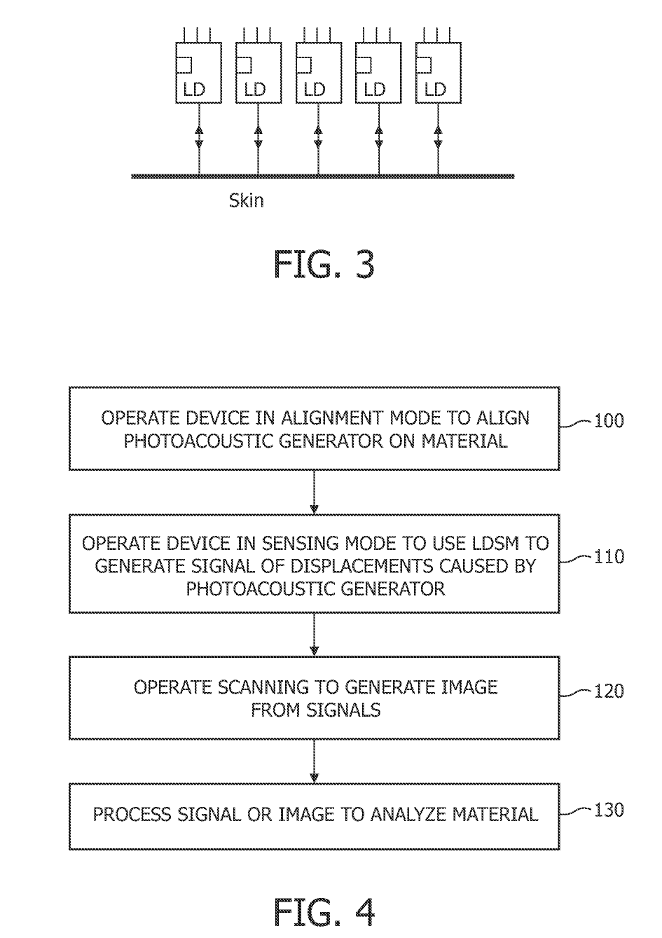

The imaging can be done in various ways. In one embodiment, the laser diode is mounted on a scanning stage and the time traces are recorded. This enables a 2D image to be obtained. In another embodiment, there is an array of laser diodes as shown in FIG. 3, to sense a line, or arranged in a 2D array to sense a line or an image with less or no need for scanning. These laser diodes can collect the data simultaneously to obtain a line or a 2D image. By avoiding part or all of the scanning procedure this can save the time required for the scan.

FIG. 4 shows method steps according to an embodiment. At step 100 the device is operated in an alignment mode to align the photo acoustic generator. This can be a manual process based on alignment information fed back to an operator, or it can be automated. The sensor may be in a fixed or movable position relative to the photo acoustic generator. At step 110 the device is operated in a sensing mode using LDSM to generate the signal of the displacements caused or stimulated by the generator. At step 120 the scanning of the sensor is carried out if appropriate to generate a line or an image. At step 130 processing of the signal in the form of discrete values or a line or an image, is carried out to analyze the material. This analyzing can involve image processing to filter an image, pick out points of interest, provide reference values or thresholds for comparison and so on, to enable an operator to deduce more information about the material.

### Implementation Example for Glucose Measurements.

A device according to an embodiment can have a housing containing a light source and a lens, the light source being controlled by a controller. A power source can be provided by an external power source to which the device is connected, or an internal source such as batteries. An operator interface can have a display screen and control buttons or other input devices coupled to the controller.

The device can optionally have some means of attachment to skin in any way, such as adhesive, or straps or other means, to maintain position during measurements. In some embodiments the area of skin being measured should have no pressure or contact by the device. The sensing part of the device should be maintained at a constant height or distance from the skin, and kept aiming at the same points on the skin.

Light from the light source can be shaped by optics into a relatively thin fan shaped beam of light and directed so that it is incident on the desired area of skin. The intensity of light in the fan beam is such that photo acoustic waves are stimulated by the light beam in tissue to a depth below the surface of the skin.

An example of a photo acoustic generator is described in U.S. Pat. No. 5,377,006, and various arrangements can be envisaged by those skilled in the art. The light beam can be generated by a laser and intensity-modulated by an acousto-optical modulator (AOM). In some embodiments, the obtained intermittent light can be expanded to a parallel beam of a desired diameter by a beam expander, which is reflected by a beam splitter or a half mirror and thereafter focused on the surface of the material to be analyzed. X or Y axis control of positioning of the beam can be controlled by movable mirrors and lenses for example. The heat distortion wave created at a focusing position on the material being analyzed generates a thermo elastic wave and also provides a minute displacement on the surface of the material. This minute displacement is detected by LDSM interferometry as explained elsewhere.

A separate light source is preferably used for the LDSM. As an added embodiment, two laser diodes in an array can be a pair of detector and a pulsed light source and by incorporating a switch, the role of detector and the source can be interchangeable. An interference intensity signal can be combined with a reference based on the output of the acousto-optical modulator, to extract only the modulated frequency and/or intensity component contained in the interference intensity signal. This frequency and/or intensity component has information relative to the surface or inside of the material being analyzed. By varying the modulated frequency and/or intensity, the thermal diffusion length can be changed and information in a direction of the depth of the material can be obtained. Changes in the material are shown as changes in the modulated frequency and/or intensity component in the interference intensity signal.

In use, the location of the point of focus can be scanned in an X direction to form the fan beam to analyze a line of the material, or moved in X and Y directions to complete an image of the material. The photo acoustic signals corresponding to the respective positions on the sample can be displayed as two-dimensional image information on a display such as a monitor.

A region of tissue in which photo acoustic waves that are detectable are stimulated is substantially bounded by the envelope of the fan beam and is referred to as the “field of view”.

The size and shape of this can be set as desired. In use the field of view can be aligned with and made larger than a blood vessel. Optionally the fan beam is configured so that at a depth of blood vessel below skin, the fan beam extends on either side of the blood vessel by an “alignment margin”. This can be equal to a few mm. So for a diameter of blood vessel equal to about 1 mm and having an expected location about 2 mm below the surface of skin, the fan beam is optionally configured so that at about 2 mm below the skin, a width of the fan beam in the plane of the fan beam is equal to or greater than about 7 mm and the fan beam has a fan angle equal to about 120 degrees.

To align the device with a blood vessel, it is placed on a region of skin below which blood vessel is expected to be located. A suitable gel or oil is optionally used to acoustically couple the device to the skin. A control signal is input to the device via the operator interface to operate in an alignment mode and the device is manually oriented so that the plane of the fan beam is substantially perpendicular to the length of the blood vessel.

The operator then moves the device back and forth substantially in a direction perpendicular to the length of blood vessel, or such scanning is carried out by a scanning mechanism. Optionally, during motion of the device, the controller controls the LDSM sensor to image features below the skin, and in particular blood vessel with ultrasound using methods known in the art. Optionally, Doppler shifted ultrasound imaging methods known in the art are used to image the blood vessel. Optionally, during motion of beam, the controller controls the light source to illuminate tissue below skin that stimulates photo acoustic waves in the tissue. Optionally, the light has at least one wavelength that is strongly absorbed by blood. Signals generated by the LDSM sensor responsive to the photo acoustic waves are used to provide a “photo acoustic” image of features such as the blood vessel. Optionally, there is an alignment control arrangement. For example the controller can determine a degree of alignment based on the ultrasound and/or photo acoustic image. This can be used to control automatic alignment of the beam, or to provide an alignment output to the operator such as an LED output and/or an audible output to a small speaker to assist manual alignment. Optionally, the controller displays the ultrasound and/or photo acoustic image on screen to facilitate aligning the device with the blood vessel.

Once the device is substantially aligned with the blood vessel, the position of the aligned device on the patient's skin is optionally marked using any suitable marking device, such as a pen for marking skin with non-toxic ink. The patient can then removes and replace the device as desired. Once properly aligned, a control signal is input to the device controller to operate in an assay mode to assay glucose in the blood vessel. In the assay mode, the controller controls the light source to illuminate the desired region with the fan beam at at least one wavelength that is scattered and/or absorbed by glucose. Signals generated responsive to photo acoustic waves generated in blood in the blood vessel by the light are used to determine concentration of glucose in the blood. Any suitable method known in the art for processing the signals to determine the glucose concentration in the blood may be used. Exemplary methods for assaying glucose in blood in blood vessels responsive to a photo acoustic effect are described in PCT publication WO 02/15776.

The present invention also includes a controller for controlling a device for analyzing a material as described above. The controller can have means for controlling photo acoustic generation of pressure waves in the material by pulsed illumination by a light source, e.g. the laser source 40. It can have means for processing a signal to analyze the material, e.g. a personal computer 60, the signal being of displacements of the material due to the pressure waves obtained from self mixing interferometry. The controller can be embodied in the personal computer or can be a separate device. The controller can be for use, for example, with a device for non invasive monitoring of blood in a human or animal body, the means for processing the signal outputting a value related the concentration of given constituents of the blood. The controller may also have means for processing the signal to provide alignment information before processing the signal to analyze the material.

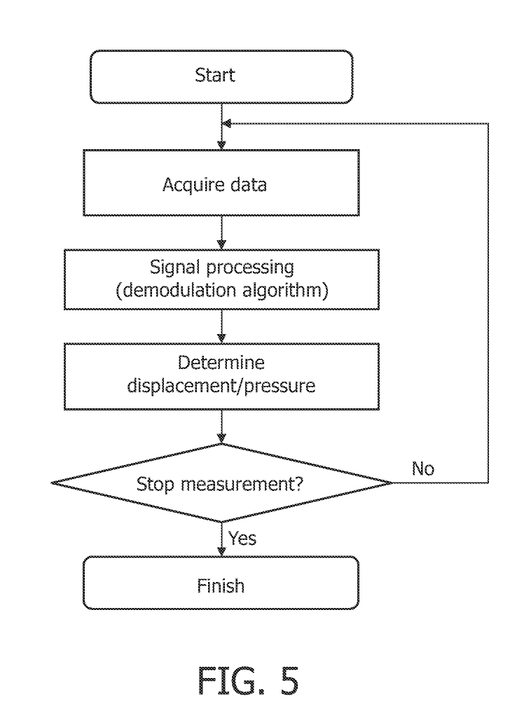

FIG. 5 is a schematic flow diagram of the signal processing that is performed in the computer 60. Computer 60 comprises a software computer program which includes code segments that when executed on a computer carry out the signal processing algorithm of FIG. 5. The measured signal is analyzed in the form of discrete values or of a line or of an image, in order to analyze the material. This analyzing can involve image processing to filter an image, pick out points of interest, provide reference values or thresholds for comparison and so on, to enable an operator to deduce more information about the material. the signal being of displacements of the material due to the pressure waves obtained from self mixing interferometry. The signal may be processed to provide alignment information before processing the signal to analyze the material. The signal processing generally starts with the acquisition of data, e.g. first of all pressure waves are generated photo acoustically by pulsed illumination by a light source and then a signal is produced using self mixing interferometry of displacements of the material due to the pressure waves. This signal is acquired at the start of the signal processing algorithm of FIG. 5. The signal is then demodulated by a demodulation algorithm and the displacements and/or pressures determined. Finally it is determined if the measurement can be stopped, and if not the signal data is acquired again.

Other variations of the devices, methods and controllers can be envisaged within the scope of the claims.

1. A device for analyzing a material, the device having a photo acoustic generator (40) for generating pressure waves in the material by pulsed illumination by a light source, and a sensor (30, 50) for producing a signal using self mixing interferometry, of displacements of the material due to the pressure waves, and a signal processor (60) for processing the signal to analyze the material.

2. The device of claim 1, the sensor comprising a laser diode (30) for outputting light which is directed towards the material so that a proportion is reflected so as to mix in a cavity (20) of the laser diode to form a modulation, the sensor also having an analyzer (50) for generating a signal representing the displacements by analyzing the modulation.

3. The device of claim 1 having an opto mechanical arrangement for scanning the sensor over an area of the material.

4. The device of claim 1, having an array of sensors for simultaneously generating signals for an area of the material.

5. The device of claim 1 having a near infra red spectroscope sensor for generating a further measurement of the material.

6. The device of claim 1, the sensor being arranged to sense parts of the material below a surface of the material.

7. The device of claim 1, the sensor being arranged to sense parts of the material on a surface of the material.

8. The device of claim 1, arranged as a non invasive monitor for monitoring concentration of constituents of blood in a human or animal body.

9. A method of analyzing a material, having the steps of photo acoustically generating pressure waves in the material by pulsed illumination by a light source, producing a signal using self mixing interferometry, of displacements of the material due to the pressure waves, and processing the signal to analyze the material.

10. The method of claim 9, having the step of generating an image of the material.

11. The method of claim 9 for non invasive monitoring of blood in a human or animal body, having the step of processing the signal to determine the concentration of given constituents of the blood.

12. The method of claim 9 having the steps of processing the signal to provide alignment information, and aligning the device on the material on the basis of the information before processing the signal to analyze the material.

13. A controller for controlling a device for analyzing a material, the controller having means for controlling photo acoustic generation of pressure waves in the material by pulsed illumination by a light source, and means for processing a signal to analyze the material, the signal being of displacements of the material due to the pressure waves obtained from self mixing interferometry.

14. The controller of claim 13 for use with a device for non invasive monitoring of blood in a human or animal body, the means for processing the signal outputting a value related the concentration of given constituents of the blood.

15. The controller, claim 13 having means for processing the signal to provide alignment information before processing the signal to analyze the material.
